(19) United States
(12) Patent Application Publication
(10) Pub. No.: US 2007/0167979 A1
(43) Pub. Date: Jul. 19, 2007

(54) Wound drain removal device

(75) Inventor: Michael Thomas Vincent Johnson, Three Forks, MT (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Bacterin International, Inc., Belgrade, MT (US)

(21) Appl. No.: 11/334,285

(22) Filed: Jan. 18, 2006

(51) Int. Cl. A61B 17/00

(52) U.S. Cl. 606/209

(57) ABSTRACT

A wound drain removal device allows an implanted wound drain to be removed from a patient in a manner that limits the amount of tension that develops within the wound drain. The wound drain removal device includes a gripping surface and a handgrip for applying a pulling force to an outflow tube of the wound drain. The gripping surface is configured to slip relative to an outer surface of the outflow tube when a removal force associated with removal of the wound drain exceeds a predetermined threshold force.

### BACKGROUND OF THE INVENTION

The present invention relates generally to wound drains. More specifically, the present invention relates to a device and method for limiting an amount of tension placed on an implanted wound drain during removal from a patient.

Wound drains are used to drain fluid from wounds and generally include a drain and an outflow tube. Wound drains are placed in a wound area during surgical wound closure to provide an avenue for tissue fluids to exit the wound. The drain resides under the skin within, or near, the wound while the outflow tubing is external to the skin and connects to a suction reservoir. Both the drain and outflow tubing are typically made of an elastic polymer such as silicone.

The conventional method for removing a wound drain from a patient is to pull on the outflow tubing until the wound drain comes out of the patient. Breakage or fragmentation of wound drains during removal from patients is a major complication of the use of wound drains for post-operative wound management. The Food and Drug Administration lists Medical Device Reports (MDR) for adverse events including those for broken wound drains and retained fragments from wound drains. A review of the MDRs indicated that approximately 74 failures per 2,000,000 wound drain removals are expected from the current paradigm of wound drain removal (a 0.004% incidence per drain).

Wound drain breakage generally occurs because either (1) the tension applied to the wound drain exceeds the tension required to separate constituents of the wound drain (e.g., the drain and outflow tubing) or (2) the wound drain was inadvertently sutured into place during wound closure. In either situation, a healthcare provider following the conventional method of pulling until the wound drain is removed from the patient will cause wound drain breakage, resulting in fragments of the wound drain being left inside the patient. These retained fragments require a second surgical exploration and necessitate additional expense and risk to the patient. These increased risks include operative morbidity and secondary wound infection.

### BRIEF SUMMARY OF THE INVENTION

The present invention is a device and method for removing an implanted wound drain from a patient. The wound drain removal device includes a gripping surface configured to slip relative to an outer surface of a wound drain outflow tube when a removal force associated with removal of the wound drain from the patient exceeds a predetermined threshold force. A handgrip is included for applying a pulling force to the wound drain outflow tube to remove the implanted wound drain from the patient.

### BRIEF DESCRIPTION OF THE DRAWINGS

FIG. 1 is a diagram of a wound drain located within a wound of a patient with an outflow tube of the wound drain extending from within the patient to the surrounding environment.

FIG. 2A is a block diagram of a wound drain removal device of the present invention used to apply a pulling force to the outflow tube of FIG. 1.

FIG. 2B is a block diagram of the wound drain removal device of FIG. 2A slipping to limit the tension of the wound drain.

FIG. 3A is side view of an embodiment of the wound drain removal device of FIG. 2A.

FIG. 3B is a front view of the wound drain removal device of FIG. 3A.

FIG. 4A is a side sectional view of the wound drain removal device of FIG. 3A used to apply a pulling force to the outflow tube of FIG. 1.

FIG. 4B is a front view of the wound drain removal device of FIG. 3A used to apply the pulling force to the outflow tube.

FIG. 5A is a side view of another embodiment of the wound drain removal device of FIG. 2A.

FIG. 5B is a front view of the wound drain removal device of FIG. 5A.

FIG. 6A is a side view of the wound drain outflow tube of FIG. 1 prior to application of a pulling force.

FIG. 6B is a side view of the wound drain outflow tube of FIG. 1 after application of the pulling force.

FIG. 7 is a graph of experimental data illustrating the inverse relationship between tension and outer diameter for a 15 French wound drain outflow tube.

FIG. 8A is a side sectional view of the wound drain removal device of FIG. 5A positioned on outflow tube of FIG. 1 prior to application of a pulling force.

FIG. 8B is a front view of the wound drain removal device of FIG. 5A positioned on the outflow tube of FIG. 1 prior to application of the pulling force.

FIG. 8C is a side sectional view of the wound drain removal device of FIG. 5A used to apply the pulling force to the outflow tube of FIG. 1.

FIG. 8D is a front view of the wound drain removal device of FIG. 5A used to apply the pulling force to the outflow tube.

FIG. 9A is a side view of another embodiment of the wound drain removal device of FIG. 2A.

FIG. 9B is a front view of the wound drain removal device of FIG. 9A.

FIG. 10A is a side sectional view of the wound drain removal device of FIG. 9A being used to apply a pulling force to the outflow tube of FIG. 1.

FIG. 10B is a side sectional view of the wound drain removal device of FIG. 10A slipping relative to the outflow tube of FIG. 1 when a tension within the outflow tube exceeds a threshold tension magnitude.

While the above-identified drawing figures set forth several embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale. Like reference numbers have been used throughout the figures to denote like parts.

### DETAILED DESCRIPTION

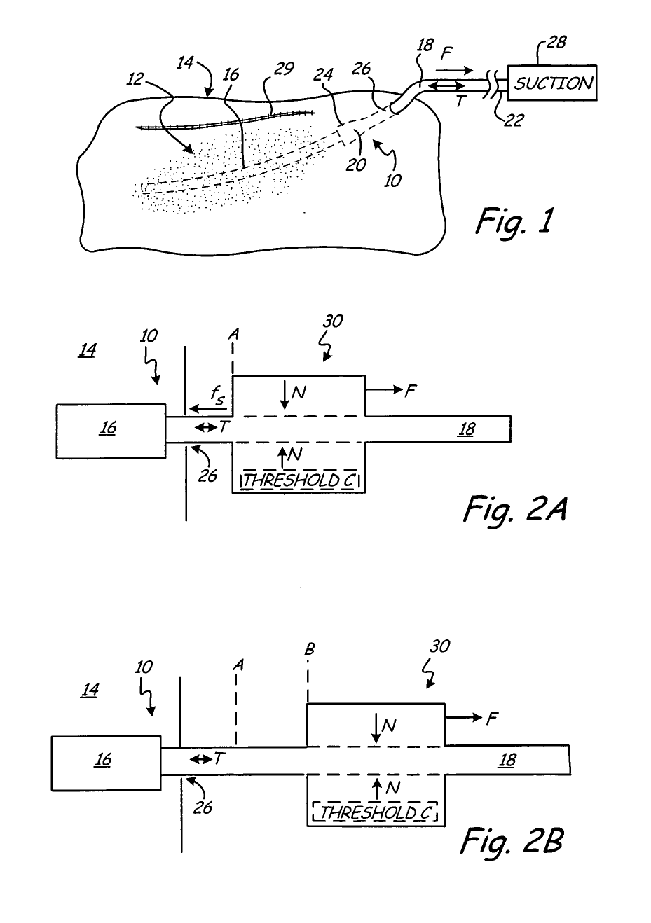

FIG. 1 is a schematic representation of wound drain 10 implanted within wound 12 of patient 14. Wound drain 10 includes drain 16 and outflow tube 18 having distal end 20 and proximal end 22. Distal end 20 of outflow tube 18 connects to wound drain 10 via connection 24.

To drain fluid from wound 12, drain 16 is located within, or adjacent, to wound 12 and is in fluid communication with outflow tube 18. Outflow tube 18 exits patient 14 via aperture 26 formed in tissue adjacent to wound 12. Proximal end 22 connects to an external suction source 28 to draw fluid from wound 12 into drain 16 and through outflow tube 18 to a reservoir associated with external suction source 28.

Methods for implanting wound drain 10 within patient 14 are well known in the art. For example, in one such method, drain 16 is positioned within, or adjacent to, wound 12. A trocar (not shown in FIG. 1) attached to proximal end 22 of outflow tube 18 is passed through tissue adjacent to wound 12 to create aperture 26. Outflow tube 18 is pulled through aperture 26 until outflow tube 18 and drain 16 are properly positioned. Wound 12 is then closed by sutures 29.

To remove wound drain 10 from patient 14 using conventional methods, a healthcare provider directly grasps outflow tube 18 by hand and pulls on outflow tube 18 to apply a pulling force F to outflow tube 18 sufficient to pull drain 16 through aperture 26 and out of patient 14. Such conventional methods may result in wound drain breakage because the healthcare provider does not have any reliable means for controlling the magnitude of tension T within wound drain 10, upon application of pulling force F.

The present invention is a device and method for controlling a magnitude of tension applied to wound drain 10 during its removal from patient 14. FIGS. 2A and 2B show a simplified block diagram of wound drain removal device 30 (hereinafter “device 30”) for use in removing wound drain 10 from patient 14. While not wishing to be bound by theory, FIGS. 2A and 2B provide an overview of some of the forces thought to be associated with removing wound drain 10 from patient 14 using device 30.

To remove wound drain 10 from patient 14, device 30 is positioned at position A relative to surface 19 of outflow tube 18, as shown in FIG. 2A. Outer surface 19 is then squeezed by device 30 via application of normal forces N. Pulling force F is applied to device 30 in a direction away from aperture 26 of patient 14, causing tension T within outflow tube 18. Slippage of device 30 along surface 19 is resisted by static friction force fₛ and, in general, device 30 remains at position A relative to surface 19 as long as pulling force F does not substantially exceed static friction force fₛ.

As discussed above, if tension T exceeds a breakage tension for wound drain 10, negative consequences can result for patient 14. Device 30 includes a predetermined threshold C to prevent tension T, upon application of pulling force F, from rising to a magnitude that may cause breakage of wound drain 10. As shown in FIG. 2B, device 30 is configured to slip relative to surface 19 and move from position A to position B when a removal force associated with removing wound drain 10 from patient 14 exceeds threshold C. In some embodiments, the removal force may comprise pulling force F or tension T.

Threshold C may be any suitable threshold to help prevent breakage of wound drain 10. In some embodiments, threshold C represents a maximum magnitude for static friction force fₛ and device 30 is configured to slip relative to surface 19 when pulling force F exceeds threshold C. Threshold C may be incorporated into device 30, for example, by including and/or controlling one or more friction properties associated with device 30. In some embodiments, threshold C represents a threshold tension magnitude for tension T and device 30 is configured to slip relative to surface 19 when tension T exceeds threshold C.

Device 30 may be located along outflow tube 18 of wound drain 10 at any suitable location for application of pulling force F. In some embodiments, device 30 is positioned on outflow tube 18 about 4 to about 6 inches away from aperture 26 of patient 14.

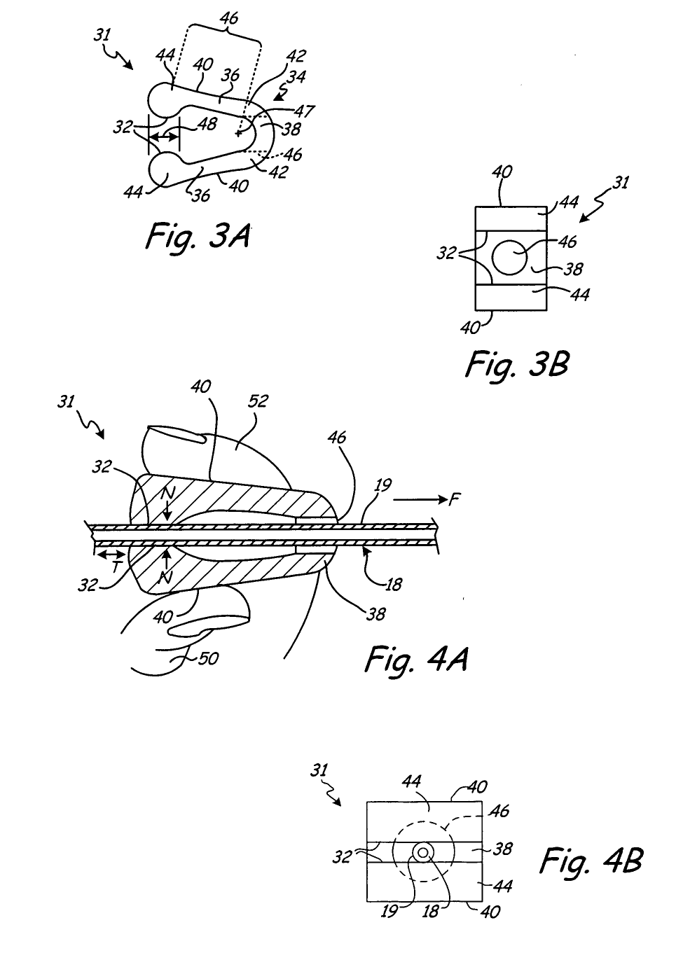

FIGS. 3A and 3B show wound drain removal device 31 (hereinafter “device 31”), with FIG. 3A showing a side view of device 31 and FIG. 3B showing a front view of device 31. Device 31 is an embodiment of device 30 of FIG. 2A. Device 31 includes a pair of gripping surfaces 32 attached to handgrip 34. Handgrip 34 includes a pair of opposing levers 36 and elastic joint 38. Each opposing lever 36 includes outer surface 40, proximal end 42, and distal end 44. Each lever 36 attaches to elastic joint 38 at proximal end 42 and one of gripping surfaces 32 at distal end 44.

Elastic joint 38 includes aperture 46, which is sized to allow for passage of outflow tube 18 of wound drain 10 there-through. When pressure is applied to outer surfaces 40 of opposing levers 36, elastic joint 38 flexes and gripping surfaces 32 move towards one another.

In one embodiment, opposing levers 36 have length 46 of about 1.0 inches as measured from fulcrum 47 to a midpoint of gripping surfaces 32, gripping surfaces 32 have width 48 of about 0.37 inches, and aperture 46 has an inner diameter of about 0.5 inches.

FIGS. 4A and 4B show device 31 of FIGS. 2A and 2B positioned on outflow tube 18 for purposes of removing wound drain 10 from patient 14. Device 31 is held between thumb 50 and forefinger 52 of a healthcare provider, with each finger exerting pressure on outer surface 40 of a different opposing lever 36. To remove wound drain 10 from patient 14, the healthcare provider squeezes device 31 between thumb 50 and forefinger 52 to apply normal forces N to outer surface 19 of outflow tube 18 with gripping surfaces 32. The healthcare provider then applies pulling force F to device 31 (and, thus, outflow tube 18) by pulling on device 31 in a direction parallel to gripping surfaces 32 and away from aperture 26 (see FIG. 1).

Gripping surfaces 32 are configured to slip relative to outer surface 19 of outflow tube 18 when pulling force F exceeds a predetermined threshold C (see FIGS. 2A and 2B). The value, or relative value, of threshold C may be determined empirically based on breakage studies of wound drain 10. Preferably, threshold C is set so that device 31 slips relative outflow tube 18 before tension T within outflow tube 18 exceeds an average breakage tension for wound drain 10. For example, for a particular wound drain 10 with an average breakage tension determined to be about 12 pounds, threshold C of device 31 may be set so that gripping surfaces 32 slip relative to outflow tube 18 when tension T is about 10 pounds. By configuring threshold C to cause gripping surfaces 32 to slip relative to outflow tube 18 when tension T has a magnitude of about 10 pounds, a buffer is provided to account for breakage variations for particular wound drains and/or healthcare providers having unusually strong handgrip strengths.

Gripping surfaces 32 may have one or more friction properties that allow gripping surfaces 32 to slip relative to outer surface 19 when pulling force F exceeds threshold C. In some embodiments, gripping surfaces 32 have a friction coefficient (e.g., a static friction coefficient) relative to outer surface 19 that allows gripping surfaces 32 to slip relative to outer surface 19 when pulling force F exceeds threshold C.

The friction coefficient of gripping surfaces 32 may be altered to achieve a desired threshold C for device 31. This may be accomplished, for example by selecting a material for gripping surfaces 32 having a suitable static friction coefficient, coating gripping surfaces 32 with a material having a suitable static friction coefficient, and/or modifying the surface characteristics of gripping surfaces 32 (e.g., texture, electrostatic properties, etc.). In addition, length 46, width 48, the elasticity of elastic joint 38, and/or the materials selected for one or more parts of device 31 may be altered to achieve a desired threshold C.

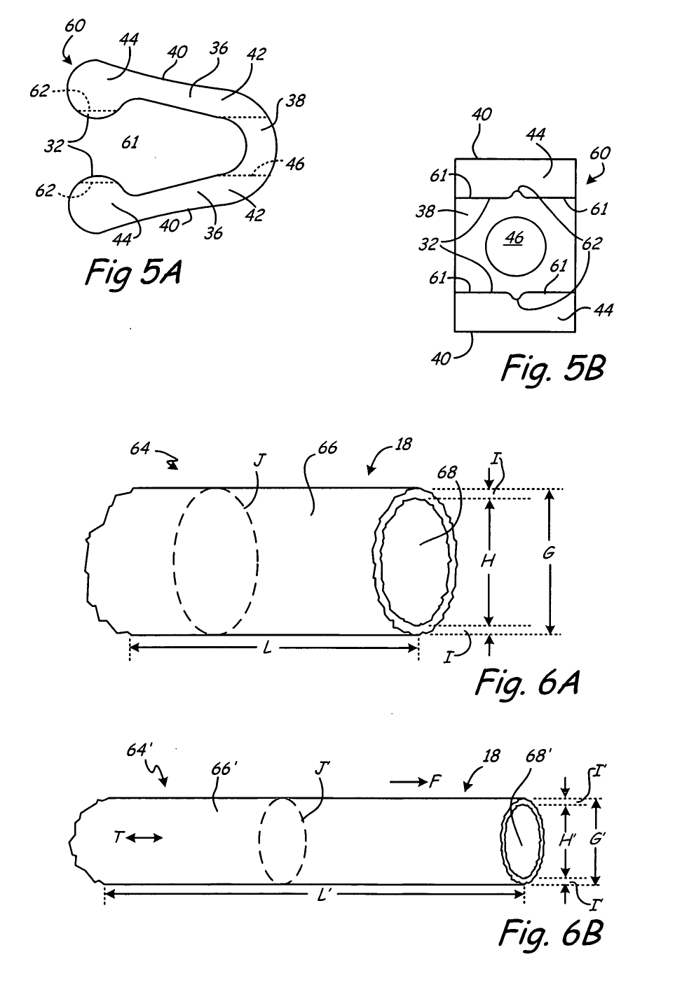

FIGS. 5A and 5B show another embodiment of device 30 of the present invention, with FIG. 5A showing a side view of wound drain removal device 60 (hereinafter “device 60”) and FIG. 5B showing a front view of device 60. Device 60 is similar to device 31 of FIGS. 3A and 3B, with the exception of gripping surfaces 32. Gripping surfaces 32 of device 60 include stop surfaces 61 and indented surfaces 62. Indented surfaces 62 are configured to slip relative to outflow tube 18 when pulling force F (see FIGS. 2A and 2B) causes tension T to exceed predetermined threshold tension magnitude C.

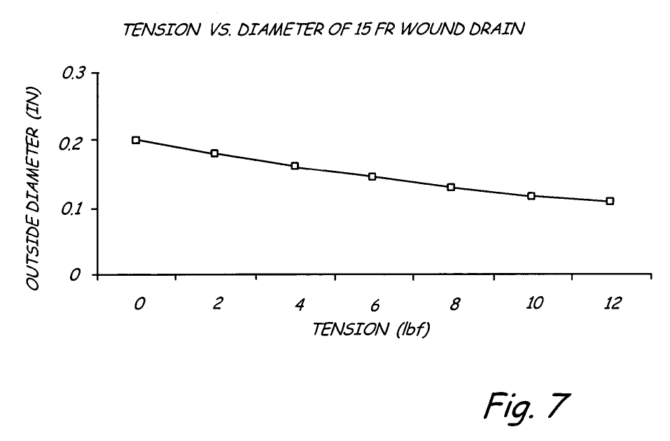

FIGS. 6A, 6B, and 7 illustrate the inverse relationship between tension T of outflow tube 18 and the outer diameter of outflow tube 18, which device 60 utilizes via indented surfaces 62 to limit the amount of tension applied to wound drain 10 during removal from patient 14.

FIG. 6A shows representative segment 64 of outflow tube 18 in a non-tensioned state. Segment 64 includes wall 66 that defines flow path 68 for transport of fluid from drain 10 to proximal end 22 of outflow tube 18 (see FIG. 1). Wall 66 is typically formed from an elastomeric polymer (e.g., such as silicone) and has outer diameter G, inner diameter H, wall thickness I, circumference J, and length L.

FIG. 6B shows representative segment 64′, which results upon application of pulling force F to segment 64. The application of pulling force F produces tension T within segment 64′, causing segment 64′ to lengthen and exhibit outer diameter G′, inner diameter H′, wall thickness I′, circumference J′, and length L′. Compared to segment 64, outer diameter G′ is smaller than outer diameter G, inner diameter H′ is smaller than inner diameter H, wall thickness I′ is thinner than wall thickness I, circumference J′ is smaller than circumference J, and length L′ is longer than length L.

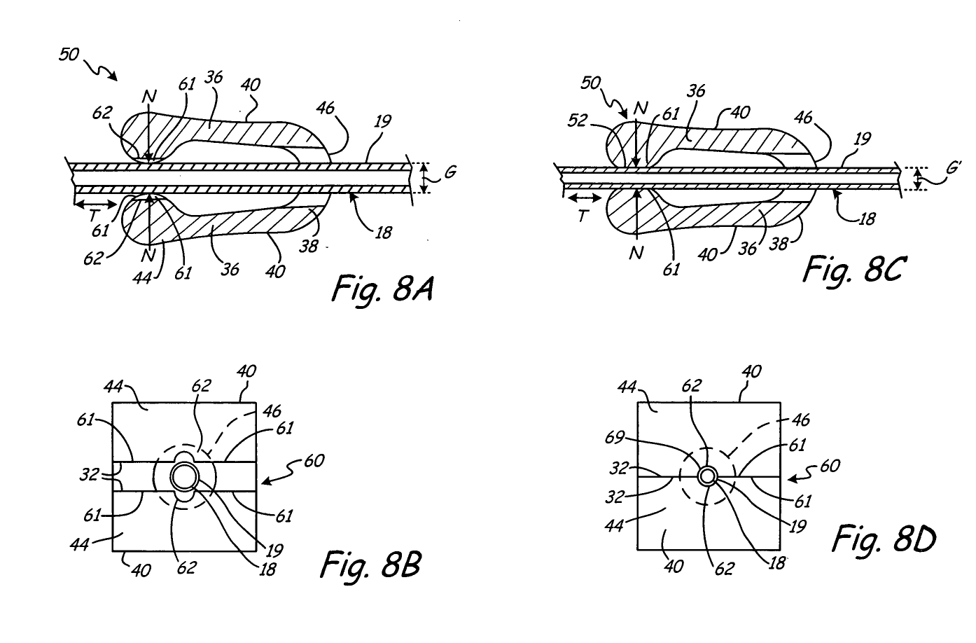

FIG. 7 illustrates graphically the inverse relationship between outer diameter G and tension T. To generate the data of FIG. 7, samples of a 15 French wound drain were tensioned using an Instron model 3343 tensile tester and outer diameter G′ at various applied forces was measured using a caliper. As indicated by the data of FIG. 7, outer diameter G′ decreases in a substantially linear manner as tension T increases. FIGS. 8A-8D illustrate how device 60 utilizes this inverse relationship to limit the magnitude of tension T during wound drain removal.

FIGS. 8A and 8B show wound drain removal device 60 (hereinafter “device 60”) positioned on outflow tube 18 prior to application of pulling force F, with FIG. 8A showing a cross-sectional side view of device 60 and outflow tube 18 and FIG. 8B showing a front view of device 60 and outflow tube 18. Outflow tube 18 is centered on indented surfaces 62 of gripping surfaces 32. Outer surfaces 40 of opposing levers 36 are squeezed to apply normal forces N to outer surface 19 of outflow tube 18. Prior to application of pulling force F outflow tube 18 is in a non-tensioned state and exhibits outer diameter G (see FIG. 6A).

FIGS. 8C and 8D show device 60 positioned on outflow tube 18 after application of pulling force F. As shown in FIGS. 8C and 8D, indented surfaces 62 form annular surface 69 when stop surfaces 61 on opposing levers 36 contact one another as shown in FIGS. 8C and 8D. Indented surface 62 are sized so that annular surface 69 defines a channel having an inner diameter that is substantially the same size as outer diameter G′ when the magnitude of Tension T equals threshold C. As shown in FIG. 8D annular surface 69 forms a cylindrical channel to coincide with the cylindrical cross-sectional profile of outflow tube 18. To facilitate this fit, indented surfaces 62 (and, thus, annular surface 69) have a curvature that is substantially the same a curvature of outer surface 19 of outflow tube 18.

In other embodiments, the cross-sectional profile of annular surface 69 may vary depending upon the cross-sectional profile of outflow tube 18. Further, as used herein, the term “annular” is defined to include not only circular or cylindrical surfaces or cross-sections, but any closed, ring-like shape, including, for example, ovals, squares, rectangles, triangles, etc. All such shapes are contemplated for use with the present invention.

Application of pulling force F to outflow tube 18 via device 60 causes tension T to develop within outflow tube 18, resulting in shrinkage of outer diameter G to outer diameter G′. As the magnitude of Tension T increases towards threshold C, outer diameter G′ approaches the diameter of annular surface 69, causing annular surface 69 to slip relative to outer surface 19 of outflow tube 18. This slippage prevents tension T from exceeding threshold C. In this embodiment, threshold C is a tension magnitude threshold. Thus, device 60 is configured to slip relative to outflow tube 18 when outer diameter G′ reaches a diameter value representative of the tension magnitude threshold.

Device 60 is but one example of wound drain removal devices of the present invention that are configured to include two or more opposing gripping surfaces (i.e., indented surfaces 62 for device 60) that are spaced from one another by a distance that is substantially the same size or larger than outer diameter G′ when outer diameter G′ reaches threshold C.

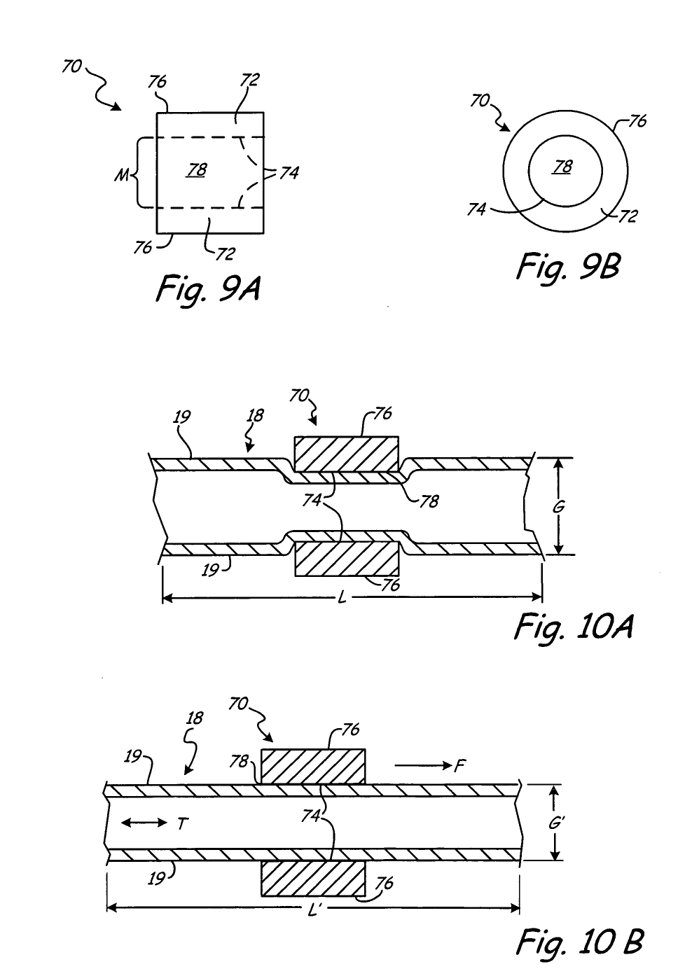

FIGS. 9A and 9B show wound drain removal device 70 (hereinafter “device 70”), which is another embodiment of the present invention that, similar to device 60, utilizes the inverse relationship between outer diameter G and tension T to prevent tension T from exceeding threshold C. FIG. 9A shows a side view of device 70 and FIG. 9B shows a front view of device 70. Device 70 includes wall 72, inner surface 74, outer surface 76, and channel 78. Inner surface 74 of wall 72 defines channel 78, which as shown in FIGS. 9A and 9B constitutes a cylindrical channel defined by inner surface 74. Channel 78 has an inner diameter M that coincides with outer diameter G′ of outflow tube 18 when tension T reaches threshold C. In other embodiments, channel 78 may have any suitable cross-sectional shape, including non-circular cross-sectional shapes.

FIGS. 10A and 10B show cross-sectional side views of device 70 positioned on outflow tube 18, with outflow tube 18 extending through channel 78 and outer surface 19 engaging inner surface 74. FIG. 10A shows outflow tube 18 prior to application of pulling force F and FIG. 10B shows outflow tube 18 after application of pulling force F. As shown in FIG. 10A, diameter M of inner surface 74 is smaller than outer diameter G of outflow tube 18.

Outer surface 76 of wall 72 functions as a handgrip for a healthcare provider to grasp onto and exert pulling force F. Outer surface 76 may have any shape suitable for use as a handgrip. Upon application of pulling force F by the healthcare provider, diameter G of outflow tube 18 reduces to diameter G′ as a function of tension T. When tension T reaches threshold C (which in this embodiment is a tension threshold magnitude), inner surface 74 slips relative to outer surface 19, preventing tension T from exceeding threshold C.

The wound drain removal devices of the present invention may be combined with one or more wound drains 10 to produce a kit or system. For example, in one embodiment, device 70 may be provided to healthcare providers in the form of a kit, with device 70 optionally pre-positioned on wound drain 10.

Thus, as described above, the present invention provides a device and method for use in removing an implanted wound drain from a patient that allows the tension within the wound drain to be controlled. The wound drain removal device includes at least one gripping surface configured to slip relative to an outflow tube of the wound drain when a predetermined threshold is exceeded.

Although the present invention has been described with reference to preferred embodiments, workers skilled in the art will recognize that changes may be made in form and detail without departing from the spirit and scope of the invention

1. A device for use in removing an implanted wound drain from a patient, the device comprising:
a handgrip for supplying a removal force to the wound drain outflow tube to remove the implanted wound drain from the patient; and
a gripping surface carried by the handgrip and configured to grip and apply the removal force to an outer surface of the wound drain outflow tube and to slip relative to the outer surface when the removal force exceeds a predetermined threshold force.

2. The device of claim 1, wherein the predetermined threshold force comprises a friction force.

3. The device of claim 2, wherein the gripping surface has one or more friction properties that allow it to slip relative to the outer surface of the wound drain outflow tube when the removal force exceeds the predetermined threshold force.

4. The device of claim 3, wherein the gripping surface has a friction coefficient relative to the outer surface of the wound drain outflow tube that allows the gripping surface to slip relative to the outer surface when the removal force exceeds the predetermined threshold force.

5. The device of claim 1, wherein the gripping surface comprises an annular surface sized to grip the outer surface of the wound drain outflow tube when the outflow tube is in a non-tensioned state and slip relative to the outer surface of the wound drain outflow tube when the removal force exceeds the predetermined threshold force.

6. The device of claim 5, wherein an inner diameter of the annular surface is smaller than an outer diameter of the outer surface of the outflow tube.

7. The device of claim 5, wherein the gripping surface comprises an inner surface of a ring and the handgrip comprises a portion of the ring.

8. The device of claim 1, wherein the device has a plurality of gripping surfaces.

9. The device of claim 8, wherein the device has two or more gripping surfaces capable of forming an annular surface.

10. The device of claim 8, wherein the handgrip comprises a pair of opposing levers and each lever is attached to a different gripping surface.

11. The device of claim 10, and further comprising:
an elastic joint attached to the pair of opposing levers.

12. A pinch grip for removing an implanted wound drain, the pinch grip comprising:
a pair of opposing gripping surfaces that are movable relative to one another;
an elastic joint; and
a pair of opposing levers attached to the elastic joint for moving the opposing gripping surfaces relative to one another and applying a force to a wound drain outflow tube;
wherein the pair of opposing gripping surfaces are configured to grip and apply pulling force to an outer surface of the wound drain outflow tube when the outflow tube has a tension less than a predetermined threshold tension and slip relative to the outflow tube when the tension exceeds the predetermined threshold tension.

13. The pinch grip of claim 12, wherein each of the opposing gripping surfaces comprises a curved gripping surface.

14. The pinch grip of claim 13, wherein a curvature of the curved gripping surface is substantially the same as an outer surface curvature of the wound drain outflow tube.

15. The pinch grip of claim 13, wherein the curved gripping surfaces are configured to form an annular surface when the pair of opposing levers are squeezed towards one another.

16. The pinch grip of claim 12, wherein the elastic joint includes an aperture sized to allow for passage of the outflow tube through the aperture.

17. A device for removing an implanted wound drain from a patient, the device comprising:
one or more gripping surfaces for applying a pulling force to a wound drain outflow tube; and
means for causing the one or more gripping surfaces to slip relative to the wound drain outflow tube when a removal force associated with removal of the wound drain from the patient exceeds a predetermined threshold.

18. A method for removing an implanted wound drain from a patient, the method comprising:
squeezing an outer surface of a wound drain outflow tube with a removal device; and
tensioning the outflow tube with the removal device to remove the implanted wound drain, wherein the removal device is configured to slip relative to the outer surface of the wound drain outflow tube when a removal force associated with removal of the wound drain from the patient reaches a predetermined threshold magnitude.

19. The method of claim 18, wherein the removal force comprises a tension of the wound drain outflow tube and the predetermined threshold magnitude comprises a threshold tension magnitude.

20. The method of claim 19, wherein the removal device comprises a pair of opposing gripping surfaces that are spaced from one another by a distance that is substantially the same size or larger than an outer diameter of the wound drain outflow tube when the tension of the wound drain outflow tube reaches the threshold tension magnitude.

21. The method of claim 18, wherein the predetermined threshold magnitude comprises a friction force and the removal force comprises a pulling force applied to the outer surface of the wound drain.

22. The method of claim 21, wherein the removal device includes a gripping surface having one or more frictional properties configured to cause the removal device to slip relative to the outer surface of the wound drain outflow tube when the pulling force exceeds the friction force.
